(19) United States
(12) Patent Application Publication
(10) Pub. No.: US 2026/0360557 A1
(43) Pub. Date: Nov. 12, 2026

(54) INTEGRATED HEALTH MONITORING SYSTEM WITH AI RECOMMENDATION ENGINE FOR AUTOMATED ANOMALY DETECTION AND ALERT TRANSMISSION FROM MULTI-DIMENSIONAL WEARABLE SENSOR DATA

(71) Applicant: SecondOpinionExpert, Inc., Westlake Village, CA (US)

(72) Inventor: Mohan ANANDA, Westlake Village, CA (US)

(21) Appl. No.: 19/672,638

(22) Filed: May 9, 2026

Related U.S. Application Data
(60) Provisional application No. 63/803,483, filed on May 9, 2025.

(51) Int. Cl. G16H 50/30; G16H 20/00; G16H 40/67; G16H 50/20

(52) U.S. Cl. CPC G16H 50/30; G16H 20/00; G16H 40/67; G16H 50/20

(57) ABSTRACT

A predictive wellness recommendation system that uses a convergence of wearable data analytics, predictive artificial intelligence, and preventive healthcare information to provide personalized, adaptive lifestyle guidance to a user that is predictive and preventive in nature for reduction lifestyle diseases. The system automatically generates notifications based on lifestyle modification predictions using artificial intelligence. The system includes a data pre-processing module receiving continuous physiological or behavioral data from wearable devices and transforming the data by data normalization scaling to a common range across heterogeneous wearable device types. An AI recommendation engine analyzes transformed data to identify trends, anomalies, or deviations from user-specific baseline health metrics. The AI recommendation engine automatically generates, in real time without manual intervention, personalized lifestyle recommendations including actions to continue, actions to avoid, or suggested medical intervention timing. A communication interface transmits time-sensitive alerts when anomalies exceed thresholds, and a digital interface presents recommendations.

### CROSS-REFERENCE TO RELATED APPLICATIONS

This application claims priority to U.S. Application No. 63/803,483, titled WELLNESS PROMPT TOOLS WITH AI-BASED PREDICTION OF LIFESTYLE MODIFICATIONS FOR WELLNESS MAINTENANCE AND DISEASE PREVENTION USING WEARABLE DEVICE DATA, filed May 9, 2025, which is hereby incorporated by reference in its entirety.

### INTRODUCTION

The present disclosure relates generally to the field of a system that communicates personalized lifestyle modifications to a user based a trained model that uses artificial intelligence (AI) or machine learning (ML).

Wearable health monitoring devices have become increasingly prevalent in recent years. Such devices may include watches, wristbands, rings, fitness trackers, and other electronic devices worn on the body that monitor and track health and fitness data. These different devices can collect various types of health-related data, including heart rate, sleep patterns, activity levels, and other vital signs.

The rising prevalence of lifestyle-related diseases and the increasing burden on healthcare systems necessitate preventive approaches rather than reactive medical interventions. Although wearable health devices collect vast amounts of health-related data, users receive fragmented feedback, which limits proactive health management.

### SUMMARY

The following presents a simplified summary of one or more aspects in order to provide a basic understanding of such aspects. The summary is not an extensive overview of all contemplated aspects and neither identifies key or critical elements of all aspects nor delineates the scope of any or all aspects. Its sole purpose is to present some concepts of one or more aspects in a simplified form as an introduction to the more detailed description that is presented later.

Aspects presented here provide relate to systems and methods for presenting users with personalized lifestyle modification recommendations that are predicted to present health issues and maintain wellness for the individual user. Aspects presented herein include the use of artificial intelligence (AI) in healthcare systems and methods for predicting personalized lifestyle modifications to prevent health issues and maintain wellness of an individual. Aspects may include using AI algorithms to interface with wearable health monitoring devices such as watches, wristbands, fitness trackers, or other electronic devices worn on the body that monitor and track health and fitness data. The AI algorithm may be used to synthesize data, and the system can then provide actionable insights to the user in a timely manner. Aspects may also include next generation AI systems that process patient medical records and other related health data to deliver accurate, high-context, and clinically relevant assessments.

Example aspects include an AI powered health application that integrates real-time user data from one or more wearable devices with user-authorized access to generate and present predictive lifestyle modifications. The application may include secure collection of multi-dimensional biometric and behavioral data and AI-based modeling to analyze trends and forecast potential health deterioration. In presenting predictive lifestyle modifications to the user, the system may generate different tiers of recommendations, e.g., including an identification of effective current habits to continue, an identification of activities or behaviors linked to negative trends that the user is recommended to avoid, and/or an identification of timely medical consultations or intervention alert for which the user is recommended to take urgent action. Aspects may include adaptive learning based on user compliance and outcomes.

The foregoing general description of the illustrative embodiments and the following detailed description thereof are merely exemplary aspects of the teachings of this disclosure and are not restrictive. Additional advantages and novel features of aspects of the present invention will be set forth in part in the description that follows, and in part will become more apparent to those skilled in the art upon examination of the following or upon learning by practice thereof.

### BRIEF DESCRIPTION OF THE DRAWINGS

The accompanying drawings, which are incorporated into and constitute a part of this specification, illustrate one or more example aspects of the present disclosure and, together with the detailed description, serve to explain their principles and implementations.

FIG. 1 is a diagram illustrating example aspects of a wellness recommendation system, in accordance with various aspects of the present disclosure.

FIG. 2 illustrates an example exchange of data and communication between one or more users and a wellness recommendation system, in accordance with various aspects of the present disclosure.

FIG. 3 is a flowchart illustrating a method for wellness recommendation using a trained model, in accordance with various aspects of the present disclosure.

FIG. 4 is a block diagram of a computer system on which the disclosed system and method can be implemented, in accordance with various aspects of the present disclosure.

FIG. 5 illustrates an example network environment in which the insurability evaluation system may be implemented, in accordance with various aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

A detailed description of systems, devices, and methods consistent with embodiments of the present disclosure is provided below. While several embodiments are described, it should be understood that disclosure is not limited to any one embodiment, but instead encompasses numerous alternatives, modifications, and equivalents. In addition, while numerous specific details are set forth in the following description in order to provide a thorough understanding of the embodiments disclosed herein, some embodiments can be practiced without some or all of these details. Moreover, for the purpose of clarity, certain technical material that is known in the related art has not been described in detail in order to avoid unnecessarily obscuring the disclosure.

The rising prevalence of lifestyle-related diseases and the increasing burden on healthcare systems necessitate preventive approaches rather than reactive medical interventions. Although wearable health devices collect vast amounts of health-related data, actionable synthesis and prediction models are lacking. Users receive fragmented feedback, which limits proactive health management. The aspects presented herein provide AI tools to assist users in lifestyle and healthcare by providing systems and methods for predicting personalized lifestyle modifications to prevent health issues and maintain wellness. The system uses AI algorithms to interface with wearable health monitoring devices to synthesize data and provide actionable insights to the user in an accessible manner.

Wearable health monitoring devices may include watches, wristbands, rings, belts, fitness trackers, or other electronic devices worn on the body that monitor and track health and fitness data. As an example of a wearable is an Apple® watch. A watch type wearable may include a health kit framework that collects heart rate, sleep, activity, and vitals data. A watch may issue alerts for an abnormal heart rate or when a hear rate falls. However, the system does not provide recommendations for long-term wellness. As presented herein, the system 102 may use wearable health metrics received from a wearable device to perform robust predictive module and provide recommendations for long term wellness. As another example of a wearable, a Fitbit® may offer health tracking and measures of health metrics. Such measurements may indicate stress or atrial fibrillation. Other examples of wearables include a WHOOP Strap®, Garmin®, and Oura Ring® that may provide a user with recovery scores, sleep analysis, and strain monitoring. While wearable devices can collect substantial amounts of health-related data, users may receive fragmented feedback from these devices. For example, a fitness tracker may provide information about step counts and heart rate, separately from other health data. A device may offer stress management features or basic health alerts, but not predictive analytics for long-term wellness planning. As presented herein, a wellness recommendation system can receive such health metrics and provide a multi-dimensional synthetization of the health data to make long term personalized lifestyle recommendations that are predicted to increase long term health of the user.

The synthesis of multi-dimensional health data from wearable devices presents various challenges. For instance, combining sleep data, activity levels, heart rate variability, and other metrics to generate actionable wellness recommendations may involve complex data processing and analysis. Additionally, providing personalized recommendations that account for individual user characteristics and health goals may involve consideration of numerous factors.

Aspects presented herein provide a system that receives user data from various wearables and provides an integrated AI model that provides predictive intervention timelines or broad lifestyle suggestions. The system presented herein provides improvements over an AI wellness application that relies on user input of symptoms, because the ongoing reception of health metrics assessed by a wearable provides a more accurate and consistent measure of user health and allows real time assessment and recommendations for wellness and improved health. Aspects presented herein provide a robust integration with real-time physiological data from wearables and enable holistic wellness maintenance tools, e.g., in addition to diagnostics.

Existing wearable health monitoring systems face significant technical challenges in synthesizing multi-dimensional biometric and behavioral data to provide actionable wellness guidance. Conventional systems may collect data from wearable devices but lack the technical capability to integrate real-time physiological data streams with predictive analytics in a manner that enables timely intervention recommendations. The fragmented nature of existing feedback mechanisms results in delayed or incomplete health assessments that fail to identify deteriorating health trends before they manifest as clinical conditions.

The wellness recommendation system described herein provides a technical improvement to health monitoring technology by implementing an AI recommendation engine that processes pre-processed wearable data to identify trends, anomalies, and deviations from baseline health metrics in real time. The technical improvement is achieved through the specific architecture of the AI recommendation engine, which includes a wellness prediction module that uses supervised learning to predict lifestyle risk factors, an anomaly detection module that identifies deviations from normal baselines, and an urgency indicator module that determines and outputs timing for medical intervention alerts.

The system further improves the technical field of health monitoring and increases accuracy of predictive medical intervention systems by implementing a feedback loop module that receives compliance information and outcome data to continuously refine the trained AI model. This adaptive learning capability enables the AI recommendation engine to improve prediction accuracy over time based on user-specific data patterns, thereby providing a technical solution that adapts to individual user characteristics rather than relying on static population-level models.

The data pre-processing module provides a technical improvement by transforming raw sensor data from heterogeneous wearable devices into a standardized format compatible with the AI recommendation engine. The pre-processing includes data normalization techniques that scale numerical values to a common range and standardize units of measurement across different data sources, enabling the AI recommendation engine to analyze data from multiple wearable device types without requiring device-specific processing logic.

The communication interface provides a technical improvement by enabling real-time transmission of push notifications, alerts, and predictive scorecards to user devices based on the analysis performed by the AI recommendation engine. For example, the push notifications may be transmitted to a mobile device, a wearable device, or another computing device associated with the user. The system automatically generates and transmits time-sensitive alerts for medical intervention timing when the urgency indicator module identifies conditions that exceed configured thresholds, thereby providing proactive remediation of potential health issues without requiring manual review by healthcare professionals.

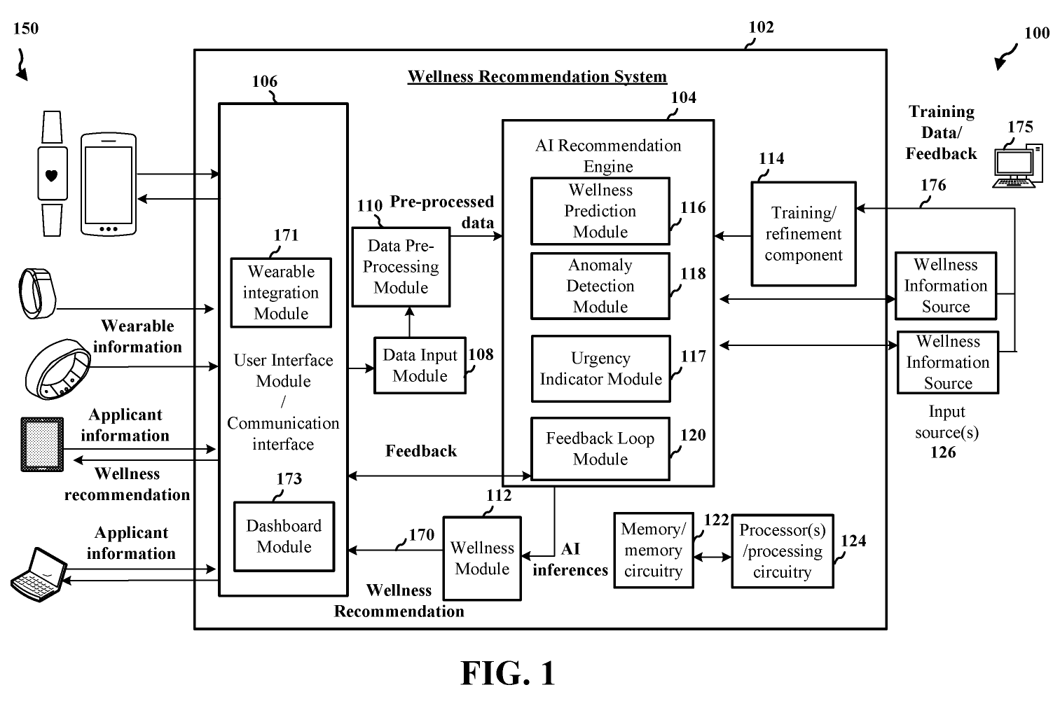

For example, the aspects presented herein provide a comprehensive system synthesizing wearable device data through AI for preventive wellness. The system described, for example in connection with FIG. 1, provides continuous, dynamic, and predictive feedback loops to provide ongoing predictions of lifestyle recommendations that may help the user to improve long term health and to continually refine the recommendations made by the system. Aspects presented herein, can suggest medical intervention timing based on cumulative indicators and provides personalization at scale.

The wellness recommendation system 102 may include a pre-processing module 110 that processes raw input data from data input module to prepare the data to prepare pre-processed data suitable to be input to the AI engine 104. For example, the pre-processing module 110 may process the raw input data using data normalization and/or natural language processing (NLP) techniques to structure the received data into a format compatible with the AI engine 104. Data normalization may include scaling numerical values to a common range, standardizing units of measurement across different data sources, and converting data types to formats compatible with the AI recommendation engine 104. In some aspects, the data normalization techniques may include min-max normalization, z-score normalization, or other statistical normalization methods to prepare the sensor data for analysis by the AI recommendation engine 104.

The data pre-processing module 110 may use natural language processing (NLP) techniques to process textual or unstructured data received from users or other sources. The NLP techniques may include tokenization, parsing, entity recognition, and semantic analysis to extract relevant information from text-based inputs. In some aspects, the data pre-processing module 110 may structure the information extracted using NLP techniques into a format that is compatible with the AI recommendation engine 104.

The sensor data or other information received from one or more wearables worn by or associated with the user, e.g., information, may be processed into a structured format that is compatible with the AI engine 104. In some aspects, the pre-processing module 110 may be configured to process vocal description or requests spoken by the user. The data pre-processing module 110 may receive audio data corresponding to a user's spoken description of health information, symptoms, or wellness goals. The data pre-processing module 110 may convert the spoken description to text using speech-to-text processing and may apply NLP techniques to extract relevant information from the converted text. The data pre-processing module 110 may then structure the information from the vocal description into a format that is compatible with the AI recommendation engine 104.

The pre-processing module 110 may remove or filter irrelevant data before inputting the processed data to the AI engine 104. For example, the data pre-processing module 110 may identify data that does not pertain to wellness analysis or that falls outside expected parameters and may exclude such data from the processed output. The filtering performed by the data pre-processing module 110 may reduce noise in the data and may improve the quality of the input provided to the AI recommendation engine 104.

In some aspects, the pre-processing module 110 may anonymize user information or user identifiers to ensure patient privacy as the patient information is input to the AI engine 104. The anonymization may include removing or replacing personally identifiable information (PII) such as names, addresses, or other identifying data with anonymized identifiers. In some cases, the data pre-processing module 110 may apply data masking, pseudonymization, or other privacy-preserving techniques to protect user information while retaining the analytical value of the data for the AI recommendation engine 104. The system may be a web-based HIPAA compliant and secure system that allows users a convenient way to register and upload their medical information and health metrics. The system is designed to ensure compliance with the HIPAA encryption standards to ensure the security and privacy of an individual's Protected Health Information (PHI). Aspects may include establishing a secure connection and/or performing a HIPAA security check.

FIG. 1 illustrates an example system architecture 100 for a wellness recommendation system 102 that provides an AI-powered health application that integrates real-time data from wearables or fitness trackers with user-authorized access to generate predictive lifestyle modifications, e.g., in accordance with various aspects of the present disclosure. The system enables AI based prediction for lifestyle modifications for wellness maintenance and disease prevention using wearable device data. As illustrated, the wellness recommendation system 102 may include memory 122 (or memory circuitry) and one or more processors 124 (or processor circuitry) configured to cause a computer system to perform the aspects described in connection with the use of the wellness recommendation system, as described herein. The processor(s)/processing circuitry 124 may be coupled to the memory/memory circuitry 122 and may execute instructions stored in the memory/memory circuitry 122 to perform data processing, AI-based analysis, and recommendation generation operations.

The system 102 includes a data input layer (e.g., data input module 108) that receives, e.g., via the communication interface 106 and/or user interface component, user data from one or more wearable devices 150 for a user. The wearable device(s) 150 may include watches, wristbands, rings, belts, fitness trackers, or other electronic devices worn on the body that monitor and track health and fitness data. In some aspects, wearable devices may include a health kit framework that collects heart rate, sleep, activity, vitals data, recovery scores, sleep analysis, and/or strain monitoring, among various examples. The communication interface 106 may also be configured to receive medical information, including medical records for the user to be considered in connection with the wearable data. The communication interface 106 may also be configured to receive user feedback information, e.g., which may be received as user feedback forms, changes in health information received via the wearable(s), user input at an application or device that is communicated to the communication interface 106. The application may include secure collection of multi-dimensional biometric and behavioral data and AI-based modeling to analyze trends and forecast potential health deterioration. The system may include a communication interface and/or user interface that enables a user to upload data. For example, the communication interface may allow the user to upload, link, or otherwise provide other health information such as medical records or information about the user. The AI engine is configured to extract relevant information, e.g., using OCR and NLP models, and contextualizes the information within the diagnostic flow.

The wellness recommendation system 102 may include a user interface module 106 that provides a user interface having one or more entry components for a user to enter health information or other user information about the user. For example, the communication interface may provide for communication between the user device 150 and system 102 over a network. For example, the user interface may include one or more entry components that allow a user to type or enter text about health information, user information, wellness goals, or to select areas of concern via a drop down box. In some aspects, the wellness recommendation system 102 may include a voice module or voice interface (e.g., as part of a user interface or communication interface) that is configured to receive voice entry of data. For example, a user may vocally describe their user information or make a verbal request for wellness recommendations. The wellness recommendation system may receive the spoken description and convert the description to text and/or process the vocal description for entry to the AI engine 104. A user may enter additional health information, health history information, family history, and/or test result information via an entry component that may allow the user to enter text, select from a set of options, vocally describe information, and/or upload or link files. In some aspects, the user interface may include an entry component that enables a user to upload files or link files/records for the patient and/or to give consent to access files for the patient.

Any document that is uploaded, or transmitted electronically to the system, will remain confidential and the system will not reveal it to anyone other than the patient and the doctors necessary for the guided diagnosis. The documents may be encrypted or may be automatically deleted after the processing is complete. In some aspects, the only individual that may have access to the user's medical data is the physician reviewing the report generated by the system.

The user interface may also be designed to present AI generated output, push notifications, alerts, scorecards, or reports, as shown at 170, for display to the user. In some aspects, the user wearing the wearable may authorize another user or establish a link with another user to receive information, such as an emergency contact, a medical professional, a wellness coach, etc. In some aspects, the wellness predictions may be sent to the user and/or one or more additional users linked or associated by the user. The wellness recommendation system 102 may include an authorized user linking feature that enables a user wearing a wearable device to authorize another user to receive information from the wellness recommendation system 102. In some aspects, the user may establish a link with an emergency contact, a medical professional, or a wellness coach to receive wellness predictions and recommendations generated by the AI recommendation engine 104. The authorized user linking feature may allow the user to specify the type of information that may be shared with each linked user and the circumstances under which the information may be transmitted. One or multiple linked users may receive push notifications, alerts, or reports based on the permissions granted by the user. As an example, one or more linked user may receive an alert when the AI recommendation engine 104 detects anomalies or deviations that exceed a threshold configured by the user.

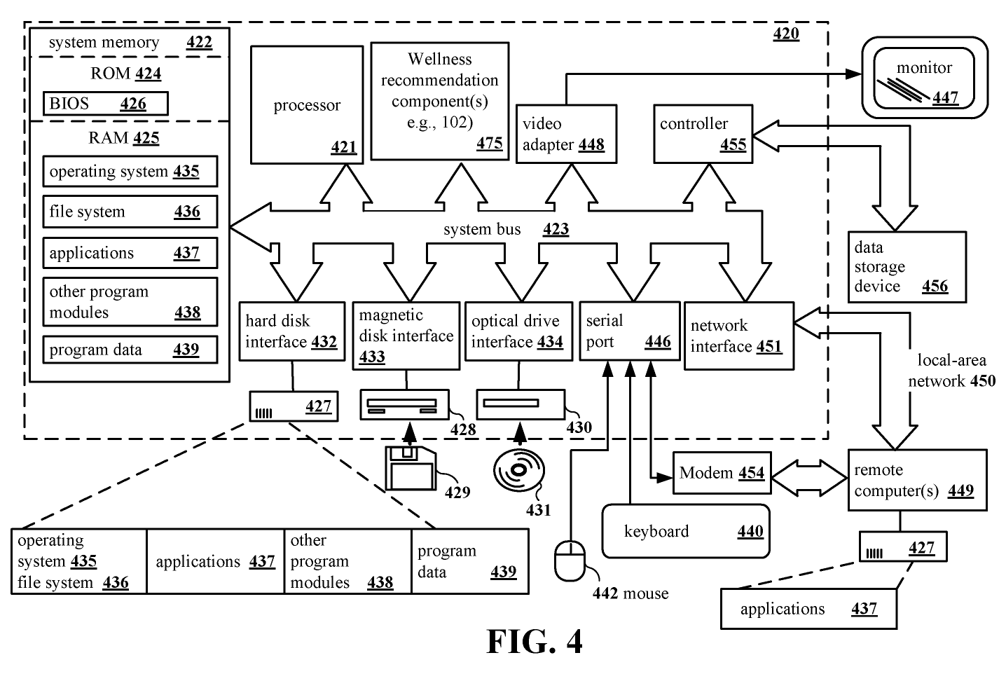

In some aspects, model refinement feedback may be provided, as shown at 176, based on prior wellness recommendations. In some aspects, the feedback may be from outside sources 126, such as medical professionals, clinicians, studies, etc. In other aspects, the feedback may be based on direct or implicit feedback from the user. The user interface module 106 may support multiple input formats such as sensor data from various wearable devices, Electronic Health Records (EHR), PDFs, among other examples. The user interface module 106 may comprise, or be comprised in, a communication interface that is configured to enable the exchange of communication via a communication path (e.g., whether a wired path, a cable path, a fiber optic path, a wireless link, and/or other communication channel between computer systems) between a user device 150 and the wellness recommendation system 102. In some aspects, the wellness recommendation system may be provided at a remote server that communicates with the user devices via the internet, e.g., such as described in connection with FIG. 4.

The wellness recommendation system 102 includes a data pre-processing unit 110 that processes data for input to a secure cloud-based AI engine using deep learning, probabilistic reasoning, and anomaly detection, e.g., the AI recommendation engine 104.

The AI recommendation engine 104 analyzes the received user input, e.g., including real-time wearable data for the user, using a trained model and outputs tiered feedback to the user. For example, the system 102 may output via the communication interface 106 push notifications to a user device, predictive scorecards to a user device, among other examples. The predictive scorecards may be updated based on new data received from the wearable device(s) 150 and may reflect changes in the user's health metrics over time. In some cases, the predictive scorecards may include trend indicators that show whether the user's wellness metrics are improving, declining, or remaining stable. In presenting predictive lifestyle modifications to the user, the system may generate different tiers of recommendations, e.g., including an identification of effective current habits to continue, an identification of activities or behaviors linked to negative trends that the user is recommended to avoid, and/or an identification of timely medical consultations or intervention alert for which the user is recommended to take urgent action. The reports may be presented via the dashboard module 173 or may be transmitted to the user via electronic communication such as email or in-application messaging.

The AI-based modeling of the wellness recommendation system 102 analyzes trends and forecasts potential health deterioration. The system 102 provides longitudinal tracking of overall wellness using dynamic AI models. The system is configured to include adaptive learning based on user compliance and outcomes. The AI engine may be configured to receive updated training data and feedback to refine the AI model and improve the inferences output by the AI engine. The wellness recommendation system may track user compliance with recommendations and refine the AI engine prediction algorithm based on the user compliance.

As illustrated the communication interface 106 may include a mobile application component or dashboard module 173 that is configured to provide an application for the user to interact with the system, receive recommendations, scorecards, etc., to a user through a graphical user interface and/or to provide an API for healthcare providers for the user. The system may include a dynamic query engine that outputs suggested supplemental questions to refine or improve the predicted wellness or lifestyle recommendation.

The AI recommendation engine 104 may include multiple modules. The modules may each be based on separately trained AI models for various aspects of predictive wellness recommendation and/or may together form a single, trained AI model for predictive wellness recommendations.

The AI recommendation engine 104 may include a wellness prediction model 116 that uses supervised learning to predict lifestyle risk factors based on the input wearable data, which may be considered in connection with other user information such as medical information. The wellness prediction module 116 may analyze physiological data and behavioral data received from wearable devices to identify patterns associated with health outcomes.

The AI recommendation engine 104 may include an anomaly detection module 118 that identifies and flags deviations from normal baselines. The anomaly detection module 118 may compare received wearable data against baseline health metrics established for the user. In some aspects, the anomaly detection module 118 may detect deviations in heart rate, sleep patterns, activity levels, or other physiological metrics that fall outside expected ranges for the user. The anomaly detection module 118 may flag detected deviations for further analysis by other modules of the AI recommendation engine 104 or for notification to the user.

The AI recommendation engine 104 may include an urgency indicator module 117 that is configured to identify and output suggestions to the user for appropriate timing for medical intervention. The urgency indicator module 117 may evaluate the severity of detected anomalies or predicted health risks to determine whether medical consultation is recommended. For example, when the urgency indicator module 117 identifies an urgent wellness recommendation, the AI recommendation engine 104 may output an alert to the user, e.g., as a push notification, an alarm, a flashing light or other visual indication. In some aspects, in response to an identification of a recommendation for an urgent medical intervention, the system may output an alert to another user, such as an emergency contact for the user or a medical professional.

The AI recommendation engine 104 may include a feedback loop module 120 that receives feedback from a user, healthcare provider, and/or other sources for continuous model refinement based on user feedback and wearable data trends. The feedback loop module 120 may receive feedback from a user, a healthcare provider, and other sources. In some aspects, the feedback loop module 120 may refine the AI model based on user feedback and wearable data trends. The feedback loop module 120 may receive compliance information indicating whether the user followed recommendations generated by the AI recommendation engine 104. In some cases, the feedback loop module 120 may receive outcome information indicating changes in the user's health metrics following implementation of recommendations. The feedback loop module 120 may use the received feedback and outcome information to update the trained model and improve the accuracy of predictions output by the AI recommendation engine 104.

In some aspects, the wellness prediction module 116, the anomaly detection module 118, the urgency indicator module 117, and the feedback loop module 120 may each be implemented as separate AI models that are trained independently. Each module may be trained on datasets specific to the function performed by the module. In some cases, the separately trained modules may communicate with each other to share intermediate outputs and coordinate the generation of wellness recommendations.

In some aspects, the wellness prediction module 116, the anomaly detection module 118, the urgency indicator module 117, and the feedback loop module 120 may be implemented as components of a unified AI model. The unified AI model may be trained on a combined dataset that includes data for wellness prediction, anomaly detection, urgency determination, and feedback processing. In some cases, the unified AI model may share learned representations across the different functions performed by the modules

The communication interface 106, data input module 108, and data pre-processing module 110 are configured to receive and collect data only with informed user consent. The communication via the communication interface 106 and within the system may include fully encrypted transmission and storage. In some aspects, the communication interface 106 may support multiple input formats such as sensor data from various wearable devices, Electronic Health Records (EHR), and other data sources. The system is configured to comply with GDPR, HIPAA, and/or local data protection laws.

As an example of use, the system may receive sensor data for a user with irregular sleep patterns and may send a recommendation (e.g., via push notification, alert, or other message) for the user to modify bedtime routines. In some aspects, the user may receive alerts when stress indicators suggest a potential burnout. As another example, the received wearable data may enable the system to identify a heart rate variability trend combined with low activity that triggers the system to send a preventive alert and a recommendation for the user to consult a physician before symptoms emerge. In another example, the system may output a recommendation, e.g., via an application at a user device, for the user to continue current hydration and walking routines based on positive trends in sleep quality and/or resting heart rate.

The AI engine 104 may be built on machine learning algorithms, including deep learning and/or natural language processing. For example, the AI engine 104 may use an AI program that includes a machine learning model or an artificial neural network (ANN) model. For example, AI recommendation engine 104 may use deep learning, probabilistic reasoning, and anomaly detection to analyze trends and forecast potential health deterioration. The AI recommendation engine 104 outputs inferences based on the received information to a wellness module 112. The wellness module 112 receives AI inferences from the AI recommendation engine 104 and generates wellness recommendation outputs. In some aspects, the wellness module 112 may generate different tiers of recommendations, including an identification of effective current habits to continue, an identification of activities or behaviors linked to negative trends that the user is recommended to avoid, and an identification of timely medical consultations or intervention alerts for which the user is recommended to take action. The wellness module 112 may compile the recommendations into a report or scorecard for presentation to the user via the communication interface 106.

The AI engine 104 may include a trained AI model that is trained on a vast dataset of wearable metrics and their relation to medical outcomes or other wellness outcomes to make inferences based on the sensor data that is input by the pre-processing module 110 according to patterns or relationships determined in the data input to the AI engine 104. An inference can refer to an output of the AI engine corresponding to a determination, a prediction, an identification, a decision, a recommendation of a lifestyle modification that is predicted to improve wellness for the user and/or to help the user achieve health goals. For example, based on pattern(s) or relationship(s) identified in the wearable that is input to the AI engine, the AI engine 104 outputs a push notification, an alert, a message, or another notification about the inference for one of more of a lifestyle adjustment for the user to consider to improve long term wellness.

An ML model may include unsupervised learning, supervised learning, and/or semi-supervised learning. Terms such “AI engine,” “ML engine,” AI/ML engine,” “AI/ML model,” “ML model,” “trained ML model,” “ANN,” “model,” “algorithm,” etc. may be used interchangeably.

In some aspects, the AI engine 104 may use any of machine-learning algorithms, deep-learning algorithms, neural networks, reinforcement learning, regression, boosting, and/or advanced signal processing methods for generating predictions of lifestyle changes or other actions as preventative approaches to meet wellness goals and avoid medical interventions.

Reinforcement learning is a type of machine learning that involves the concept of taking actions in an environment in order to maximize a reward. Reinforcement learning is a machine learning paradigm. Other paradigms include supervised learning and unsupervised learning. Basic reinforcement may be modeled as a Markov decision process (MDP) with a set of environment states and agent states, as well as a set of actions of the agent. A determination may be made about a likelihood of a state transition based on an action and a reward after the transition. The action selection by an agent may be modeled as a policy. The reinforcement learning may enable the agent to learn an optimal, or nearly-optimal, policy that maximizes a reward. Supervised learning may include learning a function that maps an input to an output based on example input-output pairs, which may be inferred from a set of training data, which may be referred to as training examples. The supervised learning algorithm analyzes the training data and provides an algorithm to map to new examples.

Regression analysis may include statistical analysis to estimate the relationships between a dependent variable (e.g., an outcome variable) and one or more independent variables. Linear regression is an example of a regression analysis. Non-linear regression models may also be used. Regression analysis may include estimating, or determining, relationships of cause between variables in a dataset.

Boosting includes one or more algorithms for reducing variance or bias in supervised learning. Boosting may include iterative learning based on weak classifiers (e.g., that are somewhat correlated with a true classification) with respect to a distribution that is added to a strong classifier (e.g., that is more closely correlated with the true classification) in order to convert weak classifiers to stronger classifiers. The data weights may be readjusted through the process, e.g., related to accuracy.

Among others, examples of machine learning models or neural networks that may be included in the AI engine 104 include, for example, artificial neural networks (ANN); decision tree learning; convolutional neural networks (CNNs); recurrent neural networks (RNNs), deep learning architectures in which an output of a first layer of neurons becomes an input to a second layer of neurons; support vector machines (SVM), e.g., including a separating hyperplane (e.g., decision boundary) that categorizes data; regression analysis; diffusion models; large language models (LLMs); Bayesian networks; genetic algorithms; deep convolutional networks (DCNs) configured with additional pooling and normalization layers; and/or deep belief networks (DBNs).

The AI recommendation engine 104 may include recurrent neural networks (RNNs). RNNs may include deep learning architectures in which an output of a first layer of neurons becomes an input to a second layer of neurons. In some aspects, RNNs may process sequential data such as time-series health metrics collected from wearable devices. RNNs may maintain internal state information that allows the RNNs to capture temporal dependencies in the input data.

The AI recommendation engine 104 may include support vector machines (SVMs). SVMs may include a separating hyperplane that categorizes data. In some aspects, SVMs may classify health data into different risk categories based on input features extracted from wearable device data.

The AI recommendation engine 104 may include diffusion models. Diffusion models may generate data by learning to reverse a gradual noising process. In some aspects, diffusion models may be used to generate synthetic health data for training or to model probability distributions of health outcomes.

The AI recommendation engine 104 may include large language models (LLMs). LLMs may process and generate natural language text based on learned patterns from large text corpora. In some aspects, LLMs may be used to generate natural language wellness recommendations or to process textual health information provided by users.

The AI recommendation engine 104 may include Bayesian networks. Bayesian networks may represent probabilistic relationships among variables using directed acyclic graphs. In some aspects, Bayesian networks may model conditional dependencies between health factors and wellness outcomes to support probabilistic reasoning in the AI recommendation engine 104.

The AI recommendation engine 104 may include genetic algorithms. Genetic algorithms may use evolutionary principles such as selection, crossover, and mutation to search for solutions in a solution space. In some aspects, genetic algorithms may be used to optimize model parameters or to identify combinations of lifestyle factors that affect wellness outcomes.

The AI recommendation engine 104 may include deep convolutional networks (DCNs). DCNs may be configured with additional pooling layers and normalization layers compared to standard CNNs. In some aspects, the pooling layers may reduce the spatial dimensions of feature maps, and the normalization layers may stabilize training by normalizing activations across layers.

The AI recommendation engine 104 may include deep belief networks (DBNs). DBNs may include multiple layers of stochastic latent variables. In some aspects, DBNs may be trained using unsupervised pre-training followed by supervised fine-tuning. DBNs may learn hierarchical representations of input data that capture complex patterns in health metrics.

In some aspects, the AI engine 104 may include an ANN with interconnected group of artificial neurons (e.g., neuron models) as nodes. Neuron model connections may be modeled as weights, in some aspects. The AI engine 104 may provide predictive modeling, adaptive control, and other applications through training via a dataset relating to wearable data, lifestyle variations, and corresponding health outcomes. In some aspects, the AI engine 104 may include a non-linear statistical data model and/or a decision making model. Machine learning may model complex relationships between input data and output information.

The AI engine 104 may include multiple layers and/or operations that may be formed by concatenation of one or more of the referenced operations. Examples of operations that may be involved include extraction of various features of data, convolution operations, fully connected operations that may be activated or deactivated, compression, decompression, quantization, flattening, etc. The term layer may indicate an operation on input data. Weights, biases, coefficients, and operations may be adjusted in order to achieve an output closer to the target output. Weights and biases are examples of parameters of a trained machine learning model. Different layers of a machine learning model may be trained separately.

A variety of connectivity patterns, e.g., including any of feed-forward networks, hierarchical layers, recurrent architectures, feedback connections, etc., may be included in a machine learning model. Layer connections may be fully connected or locally connected. For a fully connected network, a first layer neuron may communicate an output to each neuron in a second layer. Each neuron in the second layer may receive input from each neuron in the first layer. For a locally connected network, a first layer neuron may be connected to a subset of neurons in the second layer, rather than to each neuron of the second layer. A convolutional network may be locally connected and may be configured with shared connection strengths associated with the inputs for each neuron in the second layer. In a locally connected layer of a network, each neuron in a layer may have the same, or a similar, connectivity pattern, yet having different connection strengths. Aspects of the AI engine 104 may be implemented in various types of processing circuits along with memory and corresponding instruction or code. For example, the AI engine 104 may be implemented via one or more hardware circuits, such as, such as one or more central processing units (CPUs), and/or one or more graphics processing units (GPUs). GPUs may perform parallel computations on large data arrays and may accelerate the training and inference operations of neural networks. In some cases, the AI recommendation engine 104 may be implemented using a combination of CPUs and GPUs, where CPUs manage control flow and data transfer operations while GPUs perform computationally intensive matrix operations associated with neural network layers. The AI engine and/or various modules of the AI engine 104 may be stored in memory (e.g., 122) and may be accessible to one or more processors 124 to cause the processors to perform the aspects described in connection with the wellness recommendation system 102.

The training of the AI engine 104 may be performed initially, and/or in an ongoing manner, by a training component 114. The AI engine 104 may be trained, e.g., with supervised learning, unsupervised learning, semi-supervised learning, and/or reinforcement learning. The training component 114 may be configured to provide the training or that facilitates the training of the AI engine 104. During training, the AI engine 104 may be presented with an input of sensor data or other information from various types of wearables, user information, and/or medical information or other health information that the AI engine 104 uses to compute to produce an output of an inferred recommendation for one or more lifestyle adjustments predicted to improve wellness for the user. The actual output may be compared to a target well ness output and the difference may be used to adjust parameters (e.g., weights, biases, coefficients, etc.) of the AI/ML model in order to provide an output closer to the target output. Before training, the output may not be correct or may be less accurate. A difference between the output and the target output, may be used to adjust weights of a machine learning model to align the output is more closely with the target, e.g., more closely with intended wellness metrics.

A training/refinement component 114 may use a learning algorithm that calculates a gradient vector for adjustment of the weights of the AI recommendation engine 104. The gradient vector may indicate an amount by which a difference between an output of the AI recommendation engine 104 and a target output would increase or decrease if a weight were adjusted. In some aspects, the training/refinement component 114 may use gradient descent or variants of gradient descent to iteratively adjust the weights based on the calculated gradient vector.

The training/refinement component 114 may adjust the weights, biases, or coefficients of the trained AI model until an achievable error rate stops decreasing or until the error rate has reached a target level. In some aspects, the training/refinement component 114 may monitor a loss function during training to determine when the error rate has converged. In some cases, the training/refinement component 114 may use early stopping techniques to prevent overfitting when the error rate on a validation dataset stops improving.

The training data may be collected, or input, for the training component 114 to use to train the AI engine 104. The training/refinement component 114 may gather training data by accessing and analyzing sensor data, lifestyle data, user survey data, medical data, medical research, clinical guidelines, medical literature, and medical records and corresponding outcomes. In some aspects, the training component 114 may gather training data by accessing and analyzing sensor data, lifestyle data, user survey data, medical data, medical research, clinical guidelines, medical literature, and medical records and their corresponding outcomes (e.g., including medical history, medical intervention, and/or outcome).

For example, the system may be configured to send alerts with predictions of lifestyle modifications using artificial intelligence. The system may receive physiological and behavioral data from at least one wearable device. The system may analyze the data using an AI engine to identify trends, anomalies, or deviations. The system may generate personalized lifestyle recommendations that include at least one action to continue, one action to avoid, and at least one suggested medical intervention timing. The system may the send, e.g., in push notifications, alerts, presentation at a user interface, or other manner, recommendations to a user through a digital interface. The system may include any combination of a data acquisition module configured to receive data from wearable health monitoring devices; a data processing module implementing AI algorithms to predict health outcomes and generate actionable recommendations; and/or a reporting module that delivers a summary report highlighting lifestyle suggestions, risk levels, and intervention timing. The system may collect user-authorized biometric data, use machine learning models to identify health deterioration risks, and output preventive recommendations tailored to user-specific health goals. The wearable data may be received from one or more of a watch with fitness tracker or health monitoring capability, a wristband fitness tracker, a ring, or other type of wearable. In some aspects, the AI model may include a convolutional neural network trained on historical health outcome data. In some aspects, the report sent to the user by the system may include a dynamic wellness score and risk tier. In some aspects, the system may further include a module for compliance tracking and adaptive recommendation refinement. In some aspects, the data may be encrypted and complies with healthcare privacy regulations. Aspects presented herein provide a unique convergence of wearable data analytics, predictive artificial intelligence, and preventive healthcare philosophy. Aspects of the system presented herein address a significant gap in the current digital health ecosystem by providing personalized, adaptive lifestyle guidance that is predictive and preventive in nature, with the potential to dramatically reduce the burden of lifestyle diseases. Received documents can be encrypted to protect the confidentiality of the information contained in the documents, and in some cases, the documents may be automatically deleted after processing by the AI recommendation engine 104 is complete.

In some aspects, the training component 114 may receive, gather, and/or collect training data in an ongoing manner so that the training of the AI recommendation engine 104 may continue to improve and remain current according to updated outcome and correlation information. Therefore, the training of the AI engine 104 may operate to continuously improve and/or remain current according to updated outcome and correlation information. In some aspects, the training component 114 may continuously receive such input data. In some aspects, the training component 114 may periodically receive or gather the input data for training the AI engine 104. In some aspects, the training/refinement component 114 may periodically receive or gather the input data for training the AI recommendation engine 104 at scheduled intervals. Once the AI engine 104 has been trained, further input data, as well as feedback from a medical professional (e.g., in connection with 175) that reviews the inference output by the AI engine may be used to further train, refine, prune, and/or optimize the algorithms of the AI/engine 104.

The input data for training may be collected online and offline. In some aspects, online data collection may include receiving real-time data streams from wearable devices, user applications, and connected health systems. In some aspects, offline data collection may include accessing stored datasets, historical medical records, and archived research data. The training/refinement component 114 may combine online and offline data sources to provide a comprehensive training dataset for the AI recommendation engine 104.

The training/refinement component 114 may receive model refinement feedback based on prior wellness recommendations from outside sources. In some aspects, the model refinement feedback may be provided by medical professionals who review the wellness recommendations generated by the AI recommendation engine 104. In some aspects, the model refinement feedback may be provided by clinicians who evaluate the accuracy and appropriateness of the recommendations. In some aspects, the model refinement feedback may be derived from studies that assess the effectiveness of the wellness recommendations in achieving health outcomes.

Feedback from a medical professional that reviews the inference output by the AI recommendation engine 104 may be used to further train, refine, prune, and optimize the algorithms of the AI recommendation engine 104. In some aspects, the training/refinement component 114 may receive feedback indicating whether a wellness recommendation was accurate, appropriate, or effective. The training/refinement component 114 may use the feedback to adjust the trained AI model and improve the accuracy of future predictions output by the AI recommendation engine 104.

The AI recommendation engine 104 may be configured to extract relevant information using optical character recognition (OCR) and natural language processing (NLP) models. In some aspects, the AI recommendation engine 104 may use OCR to extract text from scanned documents, images, or PDF files containing medical records or health information. In some aspects, the AI recommendation engine 104 may use NLP models to process the extracted text and identify relevant health information. The AI recommendation engine 104 may contextualize the extracted information within a diagnostic flow to support the generation of wellness recommendations.

In some aspects, the wellness recommendation system may be configured to take specific automated remedial actions based on the analysis performed by the AI recommendation engine. As an example, when the anomaly detection module 118 identifies a deviation from baseline health metrics that exceeds a configured threshold, the system may be configured to automatically generate an alert and transmit the alert to the user device without requiring manual intervention. When the urgency indicator module 117 determines that a detected anomaly indicates a condition requiring medical attention, the system may be configured to automatically adjust the recommendation urgency tier and triggers transmission of notifications to linked emergency contacts or medical professionals based on authorization settings configured by the user. The automated remedial actions which the system may be configured to perform include any of: automatically generating and transmitting time-sensitive alerts, automatically adjusting recommendation priority levels based on detected anomaly severity, and/or automatically initiating communication with linked users when health metrics indicate conditions that exceed user-configured thresholds.

The real-time processing, by the wellness recommendation system, of continuous multi-dimensional sensor data streams received from heterogeneous wearable devices involves computational operations that exceed human cognitive capabilities. The simultaneous comparison of current health metrics against user-specific baselines across multiple physiological parameters, including heart rate variability, sleep patterns, activity levels, and other vital signs, requires parallel processing of data streams that cannot be performed mentally. The automatic transmission of time-sensitive alerts to user devices and linked emergency contacts based on threshold detection occurs in real time without human intervention. The continuous refinement of the trained AI model based on feedback data and compliance information involves iterative adjustment of model parameters.

The wellness recommendation system improves over prior art systems that merely collect and display health data from wearable devices. Prior systems may receive data from wearable devices and present the data to users, but lack the technical capability to synthesize multi-dimensional data streams, identify trends and anomalies in real time, and automatically generate actionable recommendations without human intervention. The wellness recommendation system provides an integrated technical solution that processes raw sensor data through the data pre-processing module 110, analyzes the processed data using the AI recommendation engine 104 to identify trends, anomalies, and deviations, generates personalized recommendations through the wellness module 112, and automatically transmits alerts and recommendations to users through the communication interface 106. The integrated technical solution operates in real time to provide proactive health management rather than reactive data display.

The specific technical architecture of the wellness recommendation system enables the improvements described herein. The pipeline from the wearable integration module 171 through the data pre-processing module 110, the AI recommendation engine 104 with its constituent modules including the wellness prediction module 116, the anomaly detection module 118, the urgency indicator module 117, and the feedback loop module 120, and the communication interface 106 with the dashboard module 173 together form an integrated technical system that provides the technical improvements over prior art systems. The ordered combination of these components enables the system to receive raw sensor data from heterogeneous wearable devices, transform the data into a standardized format, analyze the data using trained AI models to identify health trends and anomalies, generate personalized recommendations based on the analysis, and automatically transmit alerts and recommendations to users and linked contacts based on the urgency of detected conditions.

The real-time processing of continuous multi-dimensional sensor data streams received from heterogeneous wearable devices involves computational operations that exceed human cognitive capabilities. As well, the simultaneous comparison of current health metrics against user-specific baselines across multiple physiological parameters, including heart rate variability, sleep patterns, activity levels, and other vital signs, requires parallel processing of data streams from various sensors and heterogeneous devices. The automatic transmission of time-sensitive alerts to user devices and linked emergency contacts based on threshold detection occurs in real time without human intervention provides a continuous and automatic method of remediation. The continuous refinement of the trained AI model based on feedback data and compliance information involves iterative adjustment of model parameters that continuously improves weights within the AI model based on continuous output and feedback.

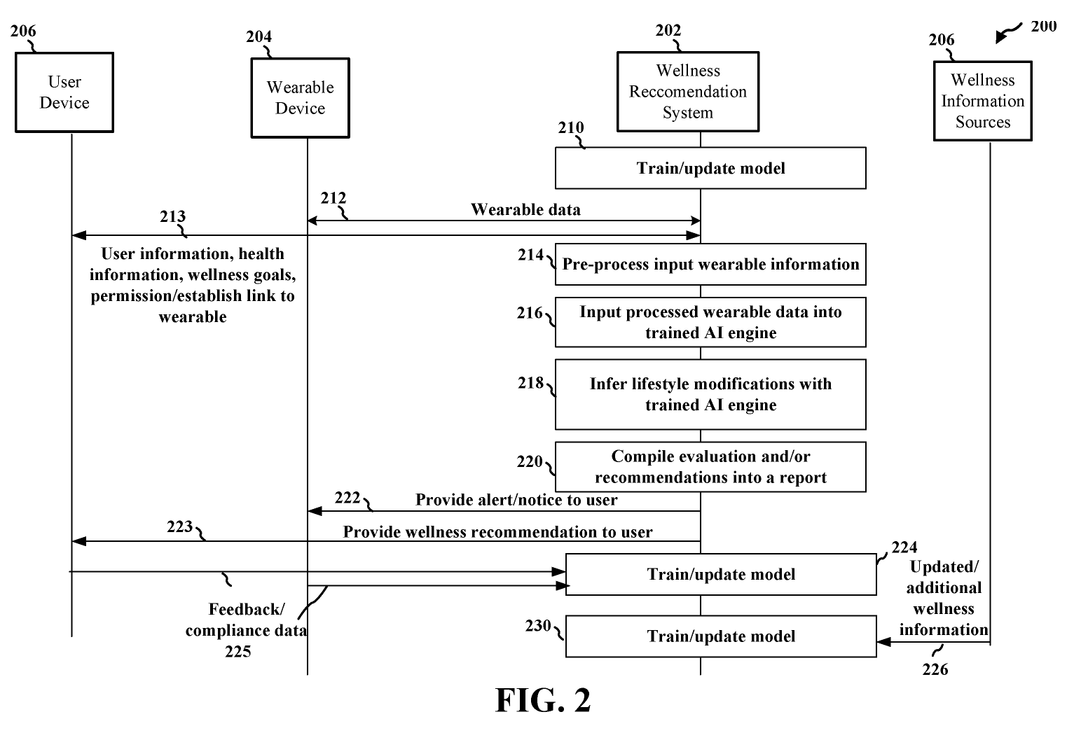

FIG. 2 is a communication flow 200 between a wellness recommendation system 202 (e.g., which may include one or more aspects of the wellness recommendation system 102 in FIG. 1), a wearable device 204 and/or a user device 206 that interacts with the wellness recommendation system 202 via a user interface, such as described in connection with FIG. 1. In some aspects, aspects performed by the user device and wearable device may be performed by a single device. wellness recommendation system 202 may include a trained AI/ML model, such as described in connection with the AI engine 104. FIG. 2 shows that the wellness recommendation system 202 may train or update the AI/ML model, or AI/ML algorithms, at 210. The training may be performed based on any combination of historical information and outcomes, among other examples.

Once the AI engine is trained, a user, e.g., via device 206, can register and consent to data use, e.g., as shown at 213. In some aspects, a user may initiate entry of information at 212, which may trigger the system to communicate with the user 206 to obtain health information and/or to receive consent to obtain such information. In some aspects, at 212, the applicant may establish a link or otherwise provide health metric information from one or more wearables, such as a stream of health data that is provided to the wellness recommendation system platform. The system 202 may import medical records via APIs and EHR integrations, e.g., based on the permission or consent provided at 213. As part of the communication exchange at 21 and/or 212, the system 202 may output questions as part of a request for user information.

The input at 212 and/or 213 may be via the user interface module 106 described in connection with FIG. 1, for example. At 214, the system 202 may pre-process the input information (e.g., the wearable sensor data) to structure the received data in a manner to be analyzed by the AI engine. At 216, the pre-processed patient data is input to the AI engine and analyzed based on the trained AI/ML model. At 218, the AI engine may provide various inferences based on the received information. For example, the system 202 evaluates the various potential health outcomes based on the received wearable data and predicts one or more lifestyle modifications that can improve wellness outcomes and/or prevent medical interventions. The system may identify tiered recommendations, e.g., as described in connection with FIG. 1. The tiered recommendations may include an identification of habits or behaviors that the user may continue, an identification of activities or behaviors that the user may avoid, and an identification of suggested timing for medical consultation or intervention. The wellness recommendation system may present the recommendations to the user through a digital interface, such as a mobile application or dashboard. If the system determines that the received user data meets a criteria, the system may send an alert, push notice, scorecard, or other message, visual, or audio indication to the user to flag or highlight the recommended action for the user. If no action is identified, the system may continue to monitor the received wearable data in an ongoing manner. In some aspects, the evaluation may provide an assessment in connection with one or multiple possible recommendation tiers, e.g., a continue tier, an avoid tier, and/or an act tier.

As examples of wellness suggestions that may be provided to the applicant at 223, the applicant may be provided with activity suggestions (e.g., walking, breathing exercises, adjustment to sleep routines, meditation, etc). A wellness suggestion may include dietary tips based on biomarkers, medication adherence reminders, and/or early warnings for abnormalities. In some aspects, geolocation-based wellness cues may be incorporated (e.g., air quality, UV index, local fitness opportunities) to provide more targeted wellness recommendations. In some aspects, a wellness recommendation may include a predictive prompt ahead of a potential medical deterioration based on pattern recognition (e.g., sleep irregularities before cardiac events).

At 220, the system 202 may compile the assessments and/or recommendations into one or more user reports or generate alerts, e.g., that are sent or displayed to the user at 204 and/or applicant 206 at 222 and/or 223. The reports may be compiled differently and or more include different information. For example, a compiled report may be displayed to the user device 206 at a user interface or may be provided in an email, text message, or other manner to the user 206. An alert may be triggered at the wearable device, such as any combination of an audio alert, a visual alert (e.g., a flashing light, a colored light, text), a vibration or motion alert, etc. As described in connection with FIG. 1, recommendations can be reported to improve health and possibly avoid long term medical interventions. Although shown as a single evaluation and report/alert, the system 202 may receive wearable data in an ongoing manner, and an ongoing analysis may be performed. Periodic reports may be provided, or the information may be continuously updated and accessible via a dashboard. In other examples, a report/alert may be triggered when an different recommendations are identified and/or when certain predicted outcomes are identified for a particular user. Although a single user is illustrated to describe the concept, the system may interact with multiple users.

Real-time alerts can be issued to the user and, if permitted, to another user such as an emergency contact or medical professional when thresholds are exceeded (e.g., abnormal heart rhythm, blood oxygen levels, etc.) to assist the user in wellness management.

The AI engine may continue to receive updated training data and feedback to refine the AI/ML model and improve the inferences output by the AI engine. For example, at 224, the system may train or update the AI/ML of the AI engine based on additional feedback or compliance information 225 received from the user. In some aspects, the AI engine may be further trained or updated, at 230 based on updated or additional wellness information 226 from various wellness information sources (e.g., 206).

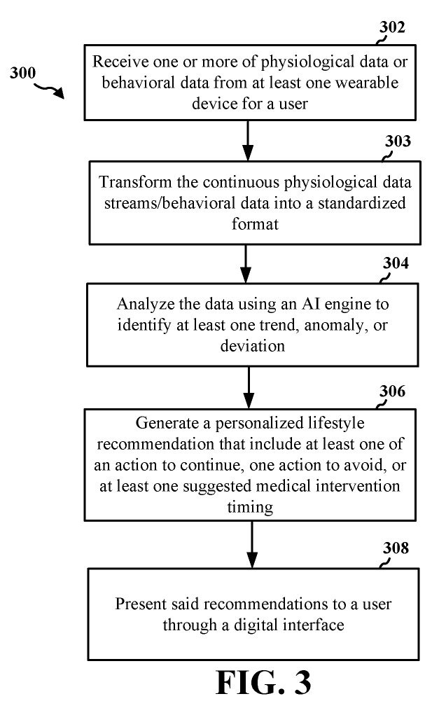

FIG. 3 is a flowchart 300 of a method of predicting and sending lifestyle modifications to improve long term wellness based on data received from a user wearable device. The method may be performed at an wellness recommendation system (e.g., 102). Some aspects of the method may be performed by an AI engine 104 or wellness recommendation component 475 of a processing system that may be part of a medical opinion system.

As shown at 302, the wellness recommendation system receives, via a user interface, one or more of physiological data or behavioral data from at least one wearable device for a user. For example, the information may be received from a user device 150 via the user interface module 106 of the wellness recommendation system 102. As an example, continuous physiological data streams and/or behavioral data may be received at a data pre-processing module from at least one wearable device of a user.

At 303, the data pre-processing module (e.g., 110) transforms the continuous physiological data streams or behavioral data into a standardized format by applying data normalization that scales numerical values to a common range across heterogeneous wearable device types, e.g., as described herein.

At 304, the wellness recommendation system analyzes the data using an AI engine to identify at least one trend, anomaly, or deviation. Example aspects of analysis are described in connection with FIGS. 1 and 2, for example. In some aspects, the AI recommendation engine may analyze the transformed data to identify at least one trend, anomaly, or deviation from user-specific baseline health metrics. As described in connection with FIG. 1, the AI recommendation engine may include a wellness prediction module that uses supervised learning to predict lifestyle risk factors, an anomaly detection module that compares received data against baseline health metrics established for the user, and an urgency indicator module that evaluates severity of detected anomalies.

At 306, the wellness recommendation system generates a personalized lifestyle recommendation that include at least one of an action to continue, one action to avoid, or at least one suggested medical intervention timing, e.g., such as described in connection with FIGS. 1 and 2. For example, the wellness recommendation system may automatically generate, by the AI recommendation engine in real time and without manual intervention, a personalized lifestyle recommendation that includes at least one of a action to continue, an action to avoid, or at least one suggested medical intervention timing;

In some aspects, the wellness recommendation may automatically transmit, by a communication interface, a time-sensitive alert to the user device when the urgency indicator determines that a detected anomaly exceeds a configured threshold.

At 308, the wellness recommendation system presents recommendations to a user through a digital interface. The presentation may include any of the aspects described in connection with FIGS. 1 and 2.

In some aspects, the wellness recommendation system may track, by a feedback loop module, user compliance indication associated with the recommendation. For example, the wellness recommendation system may receive, at the feedback loop module, outcome data indicating changes in the user’s health metrics following implementation of the recommendation, and automatically refine, by an AI training component, weight of the AI recommendation engine based on the compliance information and outcome data to adjust output prediction accuracy for the user. The AI recommendation engine may process the transformed data through an ordered combination of: the wellness prediction module outputting predictions regarding health deterioration risks, the anomaly detection module flagging deviations that fall outside expected ranges for the user, and the urgency indicator module determining whether medical consultation is recommended based on severity of detected anomalies.

In some aspects, the wellness recommendation system may collect user-authorized biometric data. In some aspects, the wellness recommendation system may use machine learning models to identify health deterioration risks. In some aspects, the wellness recommendation system may send the user a preventive recommendation tailored to user-specific health goals. In some aspects, the wearable device is selected from a group consisting of: a watch, a fitness tracker, a wristband sensor, or a ring. In some aspects, the AI engine includes a convolutional neural network trained on historical health outcome data. In some aspects, the wellness recommendation system may send the user a report that includes a dynamic wellness score and risk tier. In some aspects, the wellness recommendation system may track user compliance with the recommendation; and refine the AI engine prediction algorithm based on the user compliance. In some aspects, the wellness recommendation system may encrypt user data to comply with healthcare privacy regulations. In some aspects, the wellness recommendation system may collect user-authorized biometric data. In some aspects, the wellness recommendation system may use machine learning models to identify health deterioration risks. In some aspects, the wellness recommendation system may send a preventive recommendation message to the user tailored to user-specific health goals.

The method described in connection with FIG. 3 may further include performing any of the aspects described in connection with FIGS. 1 and 2, for example.

FIG. 4 is a block diagram illustrating a computer system 420 on which aspects of the wellness recommendation system, e.g., as described in connection with any of FIGS. 1-3 may be implemented in accordance with an example aspect. The computer system 420 can correspond to the physical server(s) on which the recommendation service (e.g., 102) is executing. In one configuration, computer system 420 may include one or more modules of a recommendation component(s) 475. The recommendation component(s) 475 and/or the computer system 420, and in particular, the file system 436 and/or the processor (e.g., 421), may be configured to perform the aspects of the flowchart in FIG. 3 or aspects of the communication flow in FIG. 2, and/or to perform the aspects described in connection with the wellness recommendation system 102.

As shown, the computer system 420 (which may comprise a personal computer or a server) includes a central processing unit 421, a system memory 422, and a system bus 423 connecting the various system components, including the memory associated with the central processing unit 421. As will be appreciated by those of ordinary skill in the art, the system bus 423 may comprise a bus memory or bus memory controller, a peripheral bus, and a local bus that is able to interact with any other bus architecture. The system memory may include permanent memory (ROM) 424 and random-access memory (RAM) 625. The basic input/output system (BIOS) 426 may store the basic procedures for transfer of information between elements of the computer system 420, such as those at the time of loading the operating system with the use of the ROM 424.

The computer system 420 may also comprise a hard disk 427 for reading and writing data, a magnetic disk drive 428 for reading and writing on removable magnetic disks 429, and an optical drive 430 for reading and writing removable optical disks 431, such as CD-ROM, DVD-ROM and other optical media. The hard disk 427, the magnetic disk drive 428, and the optical drive 430 are connected to the system bus 423 across the hard disk interface 432, the magnetic disk interface 433, and the optical drive interface 434, respectively. The drives and the corresponding computer information media are power-independent modules for storage of computer instructions, data structures, program modules, and other data of the computer system 420.

An example aspect comprises a system that uses a hard disk 427, a removable magnetic disk 429 and a removable optical disk 431 connected to the system bus 423 via the controller 455. Any type of media 456 that is able to store data in a form readable by a computer (solid state drives, flash memory cards, digital disks, random-access memory (RAM) and so on) may also be utilized.

The computer system 420 has a file system 436, in which the operating system 435 may be stored, as well as additional program applications 437, other program modules 438, and program data 439. A user of the computer system 420 may enter commands and information using keyboard 440, mouse 442, or any other input device known to those of ordinary skill in the art, such as, but not limited to, a microphone, controller, scanner, etc. Such input devices typically plug into the computer system 420 through a serial port 446, which in turn is connected to the system bus. Input devices may be also be connected in other ways, such as, without limitation, via a parallel port, a game port, or a universal serial bus (USB). A monitor 447 or other type of display device may also be connected to the system bus 423 across an interface, such as a video adapter 448. In addition to the monitor 447, the computer system may be equipped with other peripheral output devices (not shown), such as loudspeakers, a printer, etc.

Computer system 420 may operate in a network environment, using a network connection to and/or providing a user interface to one or more remote computers 449. The remote computer (or computers) 449 may be local computer workstations or servers comprising most or all of the aforementioned elements in describing the nature of a computer system 420. Other devices may also be present in the computer network, such as, but not limited to, routers, network stations, peer devices or other network nodes. The connections via a network may provide a communication interface that enables the system to receive information from multiple user and to provide wellness recommendation support, as described herein, to multiple remote users.

Network connections can form a local-area computer network (LAN) 450 and a wide-area computer network (WAN). Such networks are used in corporate computer networks and internal company networks, and they generally have access to the Internet. In LAN or WAN networks, the computer system 420 is connected to the local-area network 450 across a network adapter or network interface 451. When networks are used, the computer system 420 may employ a modem 454 or other modules that enable communications with a wide-area computer network such as the Internet. The modem 454, which may be an internal or external device, may be connected to the system bus 423 by a serial port 446. The example network connections are non-limiting examples of numerous well-understood ways of establishing a connection by one computer to another using communication modules.

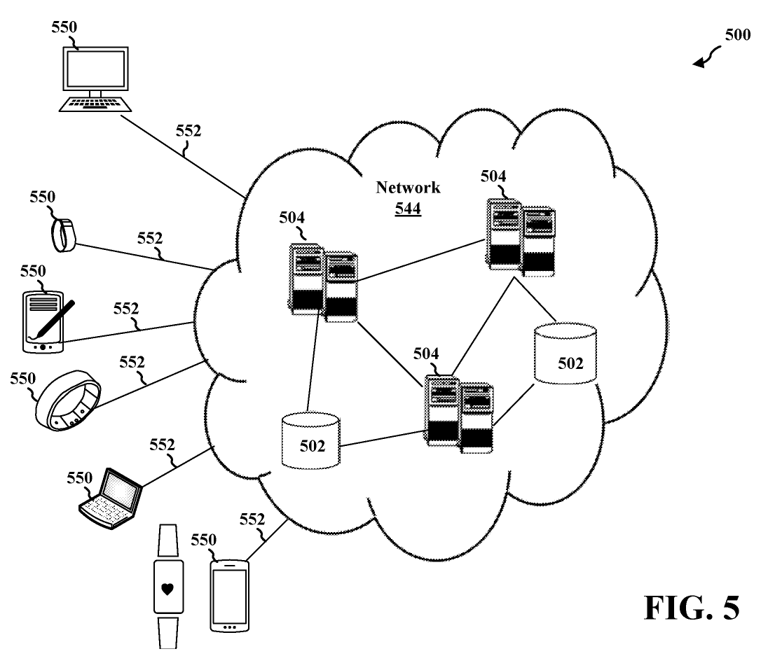

FIG. 5 shows an example communication system 500 usable in connection with aspects described herein for the recommendation system, e.g., as described in connection with any of FIGS. 1-4. The communication system 500 includes one or more accessors (also referred to interchangeably herein as one or more “users,” people, or person) and one or more terminals 550. In one aspect, data for use in accordance aspects presented herein, for example, input and/or accessed by accessors via terminals 550, such as wearables (e.g., watches, rings, wristbands, fitness trackers, etc.), telephonic devices, wireless devices, personal computers (PCs), minicomputers, mainframe computers, microcomputers, or a hand-held wireless devices coupled to one or more servers 504 such as a PC, minicomputer, mainframe computer, microcomputer, or other device having a processor and a repository for data and/or connection to a repository 502 for data, via, for example, a network 544, such as the Internet or an intranet, and couplings 552. The computer system 420 including the wellness recommendation component(s) 475 may be comprised in the network 544, e.g., including one or more servers 504 or data repositories 502. The data repositories may include storage that provides non-volatile, bulk or long term storage of data or instructions in the insurability evaluation system. The storage may take the form of a disk, tape, CD, DVD, or other reasonably high capacity addressable or serial storage medium. Multiple storage devices may be provided or available, and some include network storage or cloud-based storage.

The couplings 552 may include, for example, wired, wireless, or fiberoptic links. For example, the couplings may be part of a communications interface with the wellness recommendation system. Such a communication interface allows software and data to be transferred between the wellness recommendation system and external devices, e.g., 550. Examples of communications interfaces may include a modem, a network interface (such as an Ethernet card), a communications port, a Personal Computer Memory Card International Association (PCMCIA) slot and card, etc. Software and data transferred via communications interface can be in the form of signals , which may be electronic, electromagnetic, optical or other signals capable of being received by the communications interface. These signals may be provided to communications interface via a communications path (e.g., channel). This path carries signals and may be implemented using wire or cable, fiber optics, a telephone line, a cellular link, a radio frequency (RF) link and/or other communications channels.

In another aspect, the methods and system presented herein may operate in a stand-alone environment, such as on a single terminal.

In various aspects, the systems and methods described herein may be implemented in hardware, software, firmware, or any combination thereof. Various techniques, or certain aspects or portions thereof, may take the form of program code (i.e., instructions) embodied in tangible media, such as floppy diskettes, CD-ROMs, hard drives, a non-transitory computer readable storage medium, or any other machine-readable storage medium wherein, when the program code is loaded into and executed by a machine, such as a computer, the machine becomes an apparatus for practicing the various techniques. If implemented in software, the methods may be stored as one or more instructions or code on a non-transitory computer-readable medium. Computer-readable medium includes data storage. By way of example, and not limitation, such computer-readable medium can comprise RAM, ROM, EEPROM, CD-ROM, Flash memory or other types of electric, magnetic, or optical storage medium, or any other medium that can be used to carry or store desired program code in the form of instructions or data structures and that can be accessed by a processor of a general purpose computer.

In various aspects, the systems and methods described in the present disclosure can be addressed in terms of modules. The term “module” as used herein refers to a real-world device, component, or arrangement of components implemented using hardware, such as by an application specific integrated circuit (ASIC) or field-programmable gate array (FPGA), for example, or as a combination of hardware and software, such as by a microprocessor system and a set of instructions to implement the module’s functionality, which (while being executed) transform the microprocessor system into a special-purpose device. A module, element, or component may also be implemented as a combination of the two, with particular functions facilitated by hardware alone, and other functions facilitated by a combination of hardware and software. In particular implementations, at least a portion, and in some cases, all, of a module, element, or component may be executed on one or more processors of a general purpose computer. Accordingly, each module may be realized in a variety of suitable configurations, and should not be limited to any particular implementation or example herein. An element, or any portion of an element, or any combination of elements may be implemented as a “processing system” that includes one or more processors. When multiple processors are implemented, the multiple processors may perform the functions individually or in combination. One or more processors in a processing system may execute stored instructions, which may be referred to as software, firmware, middleware, microcode, hardware description language, or otherwise,e.g., instructions, instruction sets, code, code segments, program code, programs, subprograms, software components, applications, software applications, software packages, routines, subroutines, objects, executables, threads of execution, procedures, functions, or any combination thereof.

Components may also be implemented in software for execution by various types of processors. An identified component of executable code may, for instance, comprise one or more physical or logical blocks of computer instructions, which may, for instance, be organized as an object, a procedure, or a function. Nevertheless, the executables of an identified component need not be physically located together, but may comprise disparate instructions stored in different locations that, when joined logically together, comprise the component and achieve the stated purpose for the component.

A component of executable code may be a single instruction, or many instructions, and may even be distributed over several different code segments, among different programs, and across several memory devices. Similarly, operational data may be identified and illustrated herein within components, and may be embodied in any suitable form and organized within any suitable type of data structure. The operational data may be collected as a single data set, or may be distributed over different locations including over different storage devices, and may exist, at least partially, merely as electronic signals on a system or network. The components may be passive or active, including agents operable to perform desired functions.

While the aspects described herein have been described in conjunction with the example aspects outlined above, various alternatives, modifications, variations, improvements, and/or substantial equivalents, whether known or that are or may be presently unforeseen, may become apparent to those having at least ordinary skill in the art. Accordingly, the example aspects, as set forth above, are intended to be illustrative, not limiting. Various changes may be made without departing from the spirit and scope of the invention. Therefore, the invention is intended to embrace all known or later-developed alternatives, modifications, variations, improvements, and/or substantial equivalents. In the interest of clarity, not all of the routine features of the aspects are disclosed herein. It would be appreciated that in the development of any actual implementation of the present disclosure, numerous implementation-specific decisions must be made in order to achieve the developer’s specific goals, and these specific goals will vary for different implementations and different developers. It is understood that such a development effort might be complex and time-consuming, but would nevertheless be a routine undertaking of engineering for those of ordinary skill in the art, having the benefit of this disclosure.

Furthermore, it is to be understood that the phraseology or terminology used herein is for the purpose of description and not of restriction, such that the terminology or phraseology of the present specification is to be interpreted by the skilled in the art in light of the teachings and guidance presented herein, in combination with the knowledge of the skilled in the relevant art(s). Moreover, it is not intended for any term in the specification or claims to be ascribed an uncommon or special meaning unless explicitly set forth as such.

The various aspects disclosed herein encompass present and future known equivalents to the known modules referred to herein by way of illustration. Moreover, while aspects and applications have been shown and described, it would be apparent to those skilled in the art having the benefit of this disclosure that many more modifications than mentioned above are possible without departing from the inventive concepts disclosed herein.

Unless specifically stated otherwise, the term “some” refers to one or more. Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C. Sets should be interpreted as a set of elements where the elements number one or more. Accordingly, for a set of X, X would include one or more elements. When at least one processor is configured to perform a set of functions, the at least one processor, individually or in any combination, is configured to perform the set of functions. Accordingly, each processor of the at least one processor may be configured to perform a particular subset of the set of functions, where the subset is the full set, a proper subset of the set, or an empty subset of the set. A processor may be referred to as processor circuitry. A memory / memory module may be referred to as memory circuitry.

As used herein, the phrase “based on” shall not be construed as a reference to a closed set of information, one or more conditions, one or more factors, or the like. In other words, the phrase “based on A” (where “A” may be information, a condition, a factor, or the like) shall be construed as “based at least on A” unless specifically recited differently.

Example 1 is a predictive wellness recommendation system for automatically generating a user notification based on a prediction of a lifestyle modification using artificial intelligence (AI). The system includes a data pre-processing module configured to receive one or more continuous physiological data streams or behavioral data from at least one wearable device of a user and transform the continuous physiological data streams or behavioral data into a standardized format by applying data normalization that scales numerical values to a common range across heterogeneous wearable device types. The system also includes an AI recommendation engine configured to analyze the transformed data to identify at least one trend, anomaly, or deviation from user-specific baseline health metrics. The AI recommendation engine comprises a wellness prediction module that uses supervised learning to predict lifestyle risk factors, an anomaly detection module that compares received data against baseline health metrics established for the user, and an urgency indicator module that evaluates severity of detected anomalies. The AI recommendation engine is configured to automatically generate, in real time and without manual intervention, a personalized lifestyle recommendation that includes at least one of an action to continue, an action to avoid, or at least one suggested medical intervention timing. The system further includes a communication interface configured to automatically transmit a time-sensitive alert to the user device when the urgency indicator determines that a detected anomaly exceeds a configured threshold, and a digital interface configured to present the recommendations to a user through a digital interface.

In Example 2, the predictive wellness recommendation system of Example 1 further comprises a feedback loop configured to receive user compliance indication associated with the recommendation and outcome data indicating changes in the user's health metrics following implementation of the recommendation, and an AI training component configured to automatically refine weights of the AI recommendation engine based on the compliance information and outcome data to adjust output prediction accuracy for the user.

In Example 3, the AI recommendation engine of Example 1 or 2 processes the transformed data through an ordered combination of output predictions from the wellness prediction module regarding health deterioration risks, flagged deviations identified by an anomaly detection module based on falling outside expected ranges for the user, and a determination at the urgency indicator module whether medical consultation is recommended based on severity of detected anomalies.

In Example 4, the predictive wellness recommendation system of any of Examples 1-3 further comprises a data collection module configured to collect user-authorized biometric data, wherein the AI recommendation engine is configured to use one or more machine learning models trained to identify health deterioration risks and output a user a preventive recommendation tailored to user-specific health goals.

In Example 5, the predictive wellness recommendation system of any of Examples 1-4 further includes that the wearable device from which the one or more continuous physiological data streams or behavioral data is received includes one or more of a watch, a fitness tracker, a wristband sensor, or a ring.

Example 6 is a computer-implemented method for automatically generating a user notification based on a prediction of a lifestyle modification using artificial intelligence (AI) based system. The method comprises receiving, at a data pre-processing module, one or more of continuous physiological data streams or behavioral data from at least one wearable device of a user, and transforming, by the data pre-processing module, the continuous physiological data streams or behavioral data into a standardized format by applying data normalization that scales numerical values to a common range across heterogeneous wearable device types. The method further comprises analyzing, by an AI recommendation engine, the transformed data to identify at least one trend, anomaly, or deviation from user-specific baseline health metrics, wherein the AI recommendation engine comprises a wellness prediction module that uses supervised learning to predict lifestyle risk factors, an anomaly detection module that compares received data against baseline health metrics established for the user, and an urgency indicator module that evaluates severity of detected anomalies. The method also comprises automatically generating, by the AI recommendation engine in real time and without manual intervention, a personalized lifestyle recommendation that includes at least one of an action to continue, an action to avoid, or at least one suggested medical intervention timing, automatically transmitting, by a communication interface, a time-sensitive alert to the user device when the urgency indicator determines that a detected anomaly exceeds a configured threshold, and presenting the recommendations to a user through a digital interface.

In Example 7, the method of Example 6 further comprises tracking, by a feedback loop module, user compliance indication associated with the recommendation, receiving, at the feedback loop module, outcome data indicating changes in the user's health metrics following implementation of the recommendation, and automatically refining, by an AI training component, weight of the AI recommendation engine based on the compliance information and outcome data to adjust output prediction accuracy for the user.

In Example 8, the method of Example 6 or 7 further includes that the AI recommendation engine processes the transformed data through an ordered combination of the wellness prediction module outputting predictions regarding health deterioration risks, the anomaly detection module flagging deviations that fall outside expected ranges for the user, and the urgency indicator module determining whether medical consultation is recommended based on severity of detected anomalies.

In Example 9, the method of any of Examples 6-8 further comprises collecting user-authorized biometric data, using machine learning models to identify health deterioration risks, and sending the user a preventive recommendation tailored to user-specific health goals.

In Example 10, the method of any of Examples 6-9 further includes that the wearable device is selected from a group consisting of a watch, a fitness tracker, a wristband sensor, or a ring.

In Example 11, the AI engine of any of Examples 6-10 includes a convolutional neural network trained on historical health outcome data.

In Example 12, the method of any of Examples 6-11 further comprises sending the user a report that includes a dynamic wellness score and risk tier.

In Example 13, the method of any of Examples 6-12 further comprises tracking user compliance with the recommendation and refining the AI engine prediction algorithm based on the user compliance.

In Example 14, the method of any of Examples 6-13 further comprises encrypting user data to comply with healthcare privacy regulations.

In Example 15, the method of any of Examples 6-14 further comprises collecting user-authorized biometric data, using machine learning models to identify health deterioration risks, and sending a preventive recommendation message to the user tailored to user-specific health goals.

Example 16 is a non-transitory computer-readable storage medium containing instructions that, when executed by processor circuitry cause the processor circuitry to receive, at a data pre-processing module, one or more of continuous physiological data streams or behavioral data from at least one wearable device of a user, and transform, by the data pre-processing module, the continuous physiological data streams or behavioral data into a standardized format by applying data normalization that scales numerical values to a common range across heterogeneous wearable device types. The instructions further cause the processor circuitry to analyze, by an AI recommendation engine, the transformed data to identify at least one trend, anomaly, or deviation from user-specific baseline health metrics, wherein the AI recommendation engine comprises a wellness prediction module that uses supervised learning to predict lifestyle risk factors, an anomaly detection module that compares received data against baseline health metrics established for the user, and an urgency indicator module that evaluates severity of detected anomalies. The instructions also cause the processor circuitry to automatically generate, by the AI recommendation engine in real time and without manual intervention, a personalized lifestyle recommendation that includes at least one of an action to continue, an action to avoid, or at least one suggested medical intervention timing, automatically transmitting, by a communication interface, a time-sensitive alert to the user device when the urgency indicator determines that a detected anomaly exceeds a configured threshold, and present the recommendations to a user through a digital interface.

In Example 17, the instructions in the non-transitory computer-readable storage medium of Example 16, when executed by the processor circuitry further cause the processor circuitry to track, by a feedback loop module, user compliance indication associated with the recommendation, receive, at the feedback loop module, outcome data indicating changes in the user's health metrics following implementation of the recommendation, and automatically refine, by an AI training component, weight of the AI recommendation engine based on the compliance information and outcome data to adjust output prediction accuracy for the user.

In Example 18, the non-transitory computer-readable storage medium of Example 16 or 17 further includes instructions to cause an AI recommendation engine to process the transformed data through an ordered combination of the wellness prediction module outputting predictions regarding health deterioration risks, the anomaly detection module flagging deviations that fall outside expected ranges for the user, and the urgency indicator module determining whether medical consultation is recommended based on severity of detected anomalies.

In Example 19, the instructions of any of examples, 16-18, when executed by the processor circuitry further cause the processor circuitry to collect user-authorized biometric data, use machine learning models to identify health deterioration risks, and send the user a preventive recommendation tailored to user-specific health goals.

In Example 20, the wearable device of any of examples 16-19 is selected from a group consisting of a watch, a fitness tracker, a wristband sensor, or a ring.

Example 21 is a non-transitory computer-readable storage medium containing instructions that, when executed by processor circuitry cause the processor circuitry to perform the method of any of examples 6-15.

Example 22is an apparatus that includes memory circuitry and processor circuitry coupled to the memory circuitry, and based at least in part on information stored in the memory circuitry, is configured to perform the method of any of examples 6-15.

What is claimed is:

1. A predictive wellness recommendation system for automatically generating a user notification based on a prediction of a lifestyle modification using artificial intelligence (AI), comprising:
a data pre-processing module configured to:
receive one or more continuous physiological data streams or behavioral data from at least one wearable device of a user; and
transform the one or more continuous physiological data streams or behavioral data into a standardized format by applying data normalization that scales numerical values to a common range across heterogeneous wearable device types;
an AI recommendation engine configured to analyze transformed data to identify at least one trend, anomaly, or deviation from user-specific baseline health metrics, wherein the AI recommendation engine comprises:
a wellness prediction module that uses supervised learning to predict lifestyle risk factors,
an anomaly detection module that compares received data against baseline health metrics established for the user, and
an urgency indicator module that evaluates severity of detected anomalies,
wherein the AI recommendation engine is configured to automatically generate, in real time and without manual intervention, a personalized lifestyle recommendation that includes at least one of an action to continue, an action to avoid, or at least one suggested medical intervention timing;
a communication interface configured to automatically transmit a time-sensitive alert to the user device when the urgency indicator module determines that a detected anomaly exceeds a configured threshold; and
a digital interface configured to present the time sensitive alert to a user.

2. The predictive wellness recommendation system of claim 1, further comprising:
a feedback loop configured to receive:
user compliance indication associated with the recommendation, and
outcome data indicating changes in a user’s health metrics following implementation of the recommendation; and
an AI training component configured to automatically refine weights of the AI recommendation engine based on the compliance information and outcome data to adjust output prediction accuracy for the user.

3. The predictive wellness recommendation system of claim 1, wherein the AI recommendation engine processes the transformed data through an ordered combination of:
output predictions from the wellness prediction module regarding health deterioration risks,
flagged deviations identified by the anomaly detection module based on falling outside expected ranges for the user, and
a determination at the urgency indicator module whether medical consultation is recommended based on the severity of the detected anomalies.

4. The predictive wellness recommendation system of claim 1, further comprising:
a data collection module configured to collect user-authorized biometric data, wherein the AI recommendation engine is configured to use one or more machine learning models trained to identify health deterioration risks and output to the user a preventive recommendation tailored to user-specific health goals.

5. The predictive wellness recommendation system of claim 1, wherein a wearable device from which the one or more continuous physiological data streams or behavioral data is received includes one or more of:
a watch,
a fitness tracker,
a wristband sensor, or
a ring.

6. A computer-implemented method for automatically generating a user notification based on a prediction of a lifestyle modification using artificial intelligence (AI) based system, comprising:
receiving, at a data pre-processing module, one or more of continuous physiological data streams or behavioral data from at least one wearable device of a user;
transforming, by the data pre-processing module, the continuous physiological data streams or behavioral data into a standardized format by applying data normalization that scales numerical values to a common range across heterogeneous wearable device types;
analyzing, by an AI recommendation engine, transformed data from the data pre-processing module to identify at least one trend, anomaly, or deviation from user-specific baseline health metrics, wherein the AI recommendation engine comprises:
a wellness prediction module that uses supervised learning to predict lifestyle risk factors,
an anomaly detection module that compares received data against baseline health metrics established for the user, and
an urgency indicator module that evaluates severity of detected anomalies;
automatically generating, by the AI recommendation engine in real time and without manual intervention, a personalized lifestyle recommendation that includes at least one of an action to continue, an action to avoid, or at least one suggested medical intervention timing;
automatically transmitting, by a communication interface, a time-sensitive alert to the user device when the urgency indicator module determines that a detected anomaly exceeds a configured threshold; and
presenting the personalized lifestyle recommendation to a user through a digital interface.

7. The method of claim 6, further comprising:
tracking, by a feedback loop module, user compliance indication associated with the the personalized lifestyle recommendation;
receiving, at the feedback loop module, outcome data indicating changes in a user’s health metrics following implementation of the the personalized lifestyle recommendation; and
automatically refining, by an AI training component, weight of the AI recommendation engine based on compliance information and outcome data to adjust output prediction accuracy for the user.

8. The method of claim 6, wherein the AI recommendation engine processes the transformed data through an ordered combination of:
the wellness prediction module outputting predictions regarding health deterioration risks,
the anomaly detection module flagging deviations that fall outside expected ranges for the user, and
the urgency indicator module determining whether medical consultation is recommended based on the severity of the detected anomalies.

9. The method of claim 6, further comprising:
collecting user-authorized biometric data;
using machine learning models to identify health deterioration risks; and
sending the user a preventive recommendation tailored to user-specific health goals.

10. The method of claim 6, wherein the wearable device is selected from a group consisting of:
a watch,
a fitness tracker,
a wristband sensor, or
a ring.

11. The method of claim 6, wherein the AI engine includes a convolutional neural network trained on historical health outcome data.

12. The method of claim 6, further comprising:
sending the user a report that includes a dynamic wellness score and risk tier.

13. The method of claim 6, further comprising:
tracking user compliance with the recommendation; and
refining an AI engine prediction algorithm based on the user compliance.

14. The method of claim 6, further comprising:
encrypting user data to comply with healthcare privacy regulations.

15. The method of claim 6, further comprising:
collecting user-authorized biometric data;
using machine learning models to identify health deterioration risks; and
sending a preventive recommendation message to the user tailored to user-specific health goals.

16. A non-transitory computer-readable storage medium containing instructions that, when executed by processor circuitry cause the processor circuitry to:
receive, at a data pre-processing module, one or more of continuous physiological data streams or behavioral data from at least one wearable device of a user;
transform, by the data pre-processing module, the continuous physiological data streams or behavioral data into a standardized format by applying data normalization that scales numerical values to a common range across heterogeneous wearable device types;
analyze, by an AI recommendation engine, transformed data from the data pre-processing module to identify at least one trend, anomaly, or deviation from user-specific baseline health metrics, wherein the AI recommendation engine comprises:
a wellness prediction module that uses supervised learning to predict lifestyle risk factors,
an anomaly detection module that compares received data against baseline health metrics established for the user, and
an urgency indicator module that evaluates severity of detected anomalies;
automatically generate, by the AI recommendation engine in real time and without manual intervention, a personalized lifestyle recommendation that includes at least one of a action to continue, an action to avoid, or at least one suggested medical intervention timing;
automatically transmitting, by a communication interface, a time-sensitive alert to the user device when the urgency indicator module determines that a detected anomaly exceeds a configured threshold; and
present the personalized lifestyle recommendation to a user through a digital interface.

17. The non-transitory computer-readable storage medium of claim 16, wherein the instructions, when executed by the processor circuitry further cause the processor circuitry to:
track, by a feedback loop module, user compliance indication associated with the personalized lifestyle recommendation;
receive, at the feedback loop module, outcome data indicating changes in a user’s health metrics following implementation of the personalized lifestyle recommendation; and
automatically refine, by an AI training component, weight of the AI recommendation engine based on compliance information and outcome data to adjust output prediction accuracy for the user.

18. The non-transitory computer-readable storage medium of claim 16, wherein the AI recommendation engine processes the transformed data through an ordered combination of:
the wellness prediction module outputting predictions regarding health deterioration risks,
the anomaly detection module flagging deviations that fall outside expected ranges for the user, and
the urgency indicator module determining whether medical consultation is recommended based on the severity of the detected anomalies.

19. The non-transitory computer-readable storage medium of claim 16, wherein the instructions, when executed by the processor circuitry further cause the processor circuitry to:
collect user-authorized biometric data;
use machine learning models to identify health deterioration risks; and
send the user a preventive recommendation tailored to user-specific health goals.

20. The non-transitory computer-readable storage medium of claim 16, wherein a wearable device is selected from a group consisting of:
a watch,
a fitness tracker,
a wristband sensor, or
a ring.
